(19) United States
(12) Patent Application Publication
(10) Pub. No.: US 2007/0270792 A1
(43) Pub. Date: Nov. 22, 2007

(54) INTERFEROMETRIC CHARACTERIZATION OF ABLATED TISSUE

(76) Inventors: Willard Hennemann, Washington Crossing, PA (US); Donald B. Carlin, Pennington, NJ (US); Christian Toma, Orlando, FL (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/745,579

(22) Filed: May 8, 2007

Related U.S. Application Data
(60) Provisional application No. 60/746,660, filed on May 8, 2006.

(51) Int. Cl. A61B 18/18

(52) U.S. Cl. 606/41; 606/14; 606/21; 606/27; 606/33

(57) ABSTRACT

An apparatus including an ablation catheter having one or more optical probes attached, a system including such an apparatus and methods for using such an apparatus are disclosed herein. The optical probes may be utilized to monitor the depth of ablation in real-time during an ablation procedure, thus, reducing or eliminating the incidence of unintended injury to healthy or normal tissue during ablation.

### CROSS REFERENCES AND RELATED APPLICATIONS

The application claims priority to and the benefit of U.S. Provisional Application No. 60/746,660 entitled “Interferometric Characterization of Ablated Cardiac Tissue” filed May 8, 2006 hereby incorporated by reference in its entirety.

### BACKGROUND

Electrical signals that flow through the heart cause the cardiac muscles that make up the heart to contract in a regular pattern creating the “heart beat” or pulse. Normally each heart beat starts in the right atrium where a specialized group of cells called the Sinus Node generates an electrical signal that is conducted through the atria and into the ventricles via a single electrical pathway, the AV Node or AV Junction, that connects the atria to the ventricles below. This electrical signal and its conduction through the heart muscle causes the heart to contract in a coordinated fashion. In some instances, the electrical signals can become blocked or a signal will travel abnormally via an alternate pathway causing a “short circuit” that disrupts the hearts normal rhythm causing it to contract irregularly potentially disturbing the normal flow of blood.

Abnormal heart rhythms (arrhythmias) that cannot be controlled by lifestyle changes and/or medication have been treated using various techniques. One such technique, cardiac ablation, is often used to treat arrhythmias that manifest as rapid heart beats arising in the atria caused by short circuits. Arrhythmias that arise in the ventricles, such as, ventricular tachycardia, can also be treated using cardiac ablation. Irregular heartbeats that arise in the atria are commonly referred to as supraventricular tachycardia (SVT) and include a number of abnormalities such as atrial fibrillation, atrial flutter, AV nodal reentrant tachycardia, AV reentrant tachycardia, atrial tachycardia and the like.

Cardiac ablation is often effected by use of a catheterized device that delivers electromagnetic energy to specific areas of the heart. Various types of energy have been used for ablation including radio frequency (RF), ultrasound, optical or infrared radiation, and the like, and cryotherapy, which freezes the tissue. During cardiac ablation, the area of the heart where the abnormal electrical signal is originating is identified, and energy is applied to this area to cauterize the tissue and isolate the tissue that is the base cause of the arrhythmia. The abnormal electrical signal is therefore eliminated alleviating the arrhythmia. In the case of artial fibrillation, en effective treatment to eliminate these extra electrical pulses is through Pulmonary Vein Ablation (or Isolation). The rationale for this approach is that the electrical triggers that promote atrial fibrillation are often closely associated with the pulmonary veins. It has been shown in animal and clinical studies that ablating tissue at or around the ostia of the pulmonary veins, which leads to electrical isolation of the pulmonary vein, can reduce the frequency of or eliminate atrial fibrillation.

Ablation energy is commonly delivered using a steerable catheter that is inserted into the heart through veins or arteries located in the groin and navigated through appropriate blood vessels into the heart. Once the catheter has reached the heart, the precise location of the abnormal electrical signal or the ectopic focus can be identified using, for example, electrodes associated with the tip of the catheter. Upon location of the ectopic focus (the source of aberrant electrical signals), ablation of cardiac arrhythmia is typically performed by delivering radio frequency (RF) energy from an RF generator via a specially designed electrode catheter to the targeted tissue. Recently, catheters and systems that ablate tissue using extreme cold, or cryotherapy, to treat supraventricular tachycardia including atrial fibrillation have become available. Whether employing RF, cryo or other energy sources, the energy delivered (or removed in the case of cryo) is used to create a lesion in the tissue adjacent (i.e. underneath) the energy-delivering/removing catheter. By creating one or more lesions, the ectopic focus may be ablated or turned into a region of necrotic tissue, thereby eliminating the source of the aberrant electrical signals. The energy is delivered to or removed from the ectopic focus by effectors at the catheter tip and delivery generally requires the catheter tip to contact the abnormal tissue.

Although cardiac ablation devices and methods are currently available, many advances may still be made to provide improved devices and methods for ablating cardiac tissue to treat arrhythmia. Ablation is intended to cause damage to targeted regions of heart tissue with specific and limited area and depth within the heart wall to effectively eliminate or isolate the tissue responsible for generating abnormal electric signals in the heart causing the arrhythmia, while preventing collateral damage to other structures including the phrenic nerve, the esophagus, or any structure or tissue not specifically targeted.

Accordingly there is need for apparatuses and methods for dealing with the variations in the degree of ablation introduced due to local variations in tissue structure and composition and uniformity of application of energy; controlling the degree of ablation as the process is applied by monitoring the damage induced by the application of energy to a specific point or confined area continuously or with multiple iterations; providing feedback to control the damaging process manually or automatically; and monitoring the degree of ablation over an area including the treated area so that the process can be iterated to provide the degree of damage according to the protocol, e.g., damage to 50% of the desired level, 75%, 90%, then 100%, over the tissue area and limiting or avoiding damage to untargeted collateral tissues or structures.

### SUMMARY

The invention presented herein includes an apparatus including an ablation catheter and at least one optical probe wherein the at least one optical probe monitors a depth of ablation during use of the ablation catheter. In certain embodiments, low coherence interferometry may be used to monitor the depth of ablation.

The at least one optical probe may be mounted to the ablation catheter on an exterior circumference of the ablation catheter, embedded in the ablation catheter, or combinations thereof. In various embodiments, the at least one optical probe may deliver and collect light in a plane parallel to a central axis of the ablation catheter, and in some embodiments, the at least one optical probe delivers and collects light in a plane perpendicular to a central axis of the ablation catheter. In other embodiments, the at least one optical probe delivers and collects light in more than one plane. In yet other embodiments, the at least one optical probe may be contained in a housing other than the ablation catheter, and in certain embodiments, the at least one optical probe is a guidewire probe.

The ablation catheter of embodiments may be any type of ablation catheter known in the art and may deliver ablation energy such as, but not limited to, electrical energy, RF energy, ultrasound energy, optical, infrared, microwave, laser light, cryogenic energy and combinations thereof.

In some embodiments, a balloon may be disposed on an exterior surface of the ablation catheter.

The invention also includes a method for monitoring ablation including providing an ablation catheter and at least one optical probe to a target area wherein the at least one optical probe monitors a depth of ablation during use of the ablation catheter, identifying a site of ablation within the target area, applying energy to the site of ablation to cause ablation, and monitoring a depth of ablation concurrently with the application of energy.

The energy applied may be any energy capable of ablating tissue including, but not limited to, electrical energy, RF energy, ultrasound energy, optical, infrared, microwave, laser light, cryogenic energy and combinations thereof.

In some embodiments, the step of monitoring uses low coherence interferometry, and in others, the step of monitoring the depth of ablation occurs in real-time. In still other embodiments, the step of monitoring may use at least one optical probe that is contained in a housing other than the ablation catheter, and in certain embodiments, the at least one optical probe may be a guidewire probe.

In various embodiment, the method may further include the step of terminating the application of energy when the injury has reached a predetermined depth. In some embodiments, the application of energy may be terminated by a user, and in others, the application of energy may be automatically terminated by a processor based on a feed-back loop controlled by the processor.

Still other embodiments of the method may further include the steps of interrogating the site of ablation following the termination of the application of energy and confirming that ablation has occurred, and in some embodiments, the method may further include interrogating the site of ablation prior to applying energy and identifying irregularities at the site of ablation.

The methods of embodiments may be used to ablate any tissue, however, in some embodiments, the ablation occurs in cardiac tissue, and in certain embodiments, the ablation may be used to treat an ectopic focus.

Further embodiments of the invention include a system for monitoring ablation including an ablation catheter, at least one optical probe, a receiver configured to receive a signal from the at least one optical probe, a processor configured to determine the depth of the ablation in real-time during ablation, and an output device for displaying the depth. In various embodiments, of the system the at least one optical probe may be used to perform low coherence interferometry.

### DESCRIPTION OF DRAWINGS

For a better understanding of the disclosure and to show how the same may be carried into effect, reference will now be made to the accompanying drawings. It is stressed that the particulars shown are by way of example only and for purposes of illustrative discussion of the preferred embodiments of the present disclosure only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 shows an embodiment of a catheter having multiple optical probes (inset) and illustrates a catheter in use probing heart tissue en-face.

FIG. 2 shows cross sections showing arrangements of a plurality of optical fiber probes associated with an ablation catheter. Panel A shows a two optical probe configuration. Panel B shows multiple optical probes arranged around an external circumference of the ablation catheter. Panel C shows multiple optical probes arranged around an external circumference of the ablation catheter and multiple optical probes embedded within the catheter. Panel D shows multiple optical probes embedded within the ablation catheter.

FIG. 3 shows an exemplary LCI output data in accordance with the present invention. Panel A shows the output data associated with healthy tissue and Panel B shows the output data associated with ablated tissue.

### DETAILED DESCRIPTION

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

It must also be noted that as used herein and in the appended claims, the singular forms “a”, “an”, and “the” include plural reference unless the context clearly dictates otherwise. Thus, for example, reference to a “lesion” is a reference to one or more lesions and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described.

The methods as described herein for use contemplate prophylactic use as well as curative use in therapy of an existing condition. As used herein, the term “about” means plus or minus 20% of the numerical value of the number with which it is being used. Therefore, about 50% means in the range of 30%-70%.

“Optional” or “optionally” means that the subsequently described structure, event or circumstance may or may not occur, and that the description includes instances where the event occurs and instances where it does not.

Generally speaking, the term “tissue” refers to any aggregation of similarly specialized cells which are united in the performance of a particular function. For example, the term “tissue” may refer to tissue that makes up an organ on which a lesion may occur.

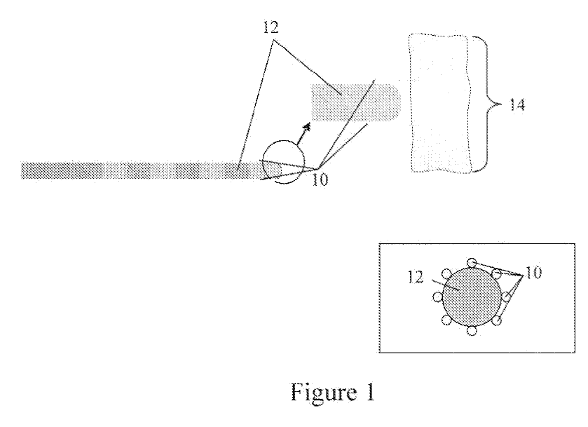

The invention described herein is generally directed to an ablation catheter having one or more optical probes which can be used to examine or interrogate tissue surrounding a lesion prior to, during, or after use of the ablation catheter. FIG. 1 illustrates an embodiment of the invention wherein ablation catheter 12 having a plurality of optical probes 10 arranged around an external surface of an ablation catheter 12 (see inset). In particular embodiments, the lesion being interrogated and the tissue ablated may be cardiac tissue or heart tissue 14, as illustrated in FIG. 1, where an abnormal electrical signal (ectopic focus) that causes an arrhythmia appears to originate. In some embodiments, the optical probe may be provided as a separate device and may therefore be contained in a housing other than that of the ablation catheter. For example, in one embodiment, at least one optical probe is a guidewire probe as described in U.S. patent application Ser. No. 11/445,514, entitled “Multi-channel Low Coherence Interferometer”, hereby incorporated by reference in its entirety.

In various embodiments, the optical probes may be used to characterize the lesion or tissue of interest using Low Coherence Interferometery (LCI) or Optical Coherence Tomography (OCT). Therefore, the optical probes may be used to deliver light to the area of interest and collect backscattered light from the tissue and may be associated with a system that further interprets the collected backscattered light and correlates it with the state of the interrogated tissue. The optical probes may be used to interrogate tissue at the distal most tip of the ablation catheter by delivering light en-face, emitting light in a plane parallel to the fiber axis and parallel to the catheter tip, or emitting light perpendicular to the fiber axis or catheter tip, in various embodiments. In other embodiments, the one or more optical probe may be built into or incorporated in the catheter or supplied to the catheter as a secondary device that fits over the catheter or is routed to the target area separately.

In other embodiments, a balloon may be positioned on the exterior of the ablation catheter as part of a balloon catheter. The balloon may be inflated during deployment to clear blood from an area surrounding the catheter tip thereby allowing an optical signal from optical probes to penetrate the tissue without scattering caused by fluid, such as blood, surrounding the tissue. In other embodiments, a balloon may be an integral element of an ablation catheter. In still other embodiments, the optical probes may make up an integral part of the balloon, so that as the balloon is inflated the one or more optical probe tips may get closer to the tissue under interrogation.

The invention further includes methods for using an ablation catheter having one or more associated optical probes wherein the optical probes provide means by which the extent of ablation injury may be monitored in real-time. In such embodiments, one or more optical probes may continually monitor an area at or near the target area to provide information regarding the depth and/or circumference of ablation injury produced by the ablation catheter. The information provided by the optical probes may be utilized by a user where the information is used to determine whether treatment with the ablation catheter should continue or stop. The information may also be supplied to a processor as part of a feed-back loop where the emission of ablation energy is terminated when the extent of injury has reached a predetermined threshold. Alternatively, the optical probes may be used to identify areas surrounding the target area where ablation has already occurred, confirm that a sufficient or desired ablation has occurred at a target area, or identify other areas of injury surrounding the target area.

Without wishing to be bound by theory, the ability to monitor the area of injury during ablation may reduce the incidence of overablation thereby preventing unintended injury to the patient. In yet other embodiments, the optical probes may be used to ensure that the ablation catheter is properly aligned with the target tissue. For example, in embodiments including several optical probes surrounding the catheter tip, the distance of the catheter from the target tissue at each point having an optical probe may be monitored allowing the user to position the probe substantially perpendicularly to the target tissue, thereby allowing an effective ablation to the targeted tissue to be more reliably delivered and unintended injury caused by applying energy from the ablation catheter at odd angles avoided.

The ablation catheter, in alternate embodiments, may be any ablation catheter known in the art. In general, an ablation catheter includes a long flexible catheter having one or more effectors attached to the distal most catheter tip. These effectors may be utilized to deliver energy, such as, for example, electrical energy, RF energy, (high intensity) ultrasound energy (HIFU), optical, infrared, microwave laser light, cryogenic energy and the like. In some embodiments, the catheter may be steerable, meaning that the shape of the catheter may be manipulated by a user to adjust the direction of travel of the catheter and/or the position of the distal most catheter tip. Ablation catheters may further include any number of sensors that may aid in the location of the target tissue. For example, in some embodiments, the catheter may include electrodes which are capable of detecting an errant electrical signal emanating from the target tissue, or a sensor capable of communicating with an external device such as an x-ray detector, an electroanatomical navigation system or an electrocardiogram.

In various embodiments, the optical probes associated with the catheter tip may be connected to an interferometer, and any type of interferometer known in the art may be used. For example, the interferometer used in embodiments of the invention may include, but not be limited to, time delay interferometers (TD-LCI), such as, scanning Michelson interferometers and autocorrelators, and optical frequency domain interferometers (OFDI), such as, spectral domain low-coherence interferometers, and these interferometers may be used to detect interference between one or more reference optical signal and one or more backscattered sample optical signal or birefringence caused by the sample. Such optical probes may be embedded in the catheter or coupled to the outermost shell of the catheter.

Optical probes of the type described above include a light source optically coupled to one or more waveguides capable of propagating an optical signal from a light source which may be located at a proximal end of the catheter to the distal most catheter tip where the light may be emitted illuminating the target. Such waveguides may further be capable of collecting backscattered light from the target and propagating the backscattered light back through the optical probe to at least one detector which may be associated with a receiver that converts backscattered light into an analog, electrical or digital signal and transmits this signal to a processor where it may be stored or interpreted.

According to various embodiments, the light source may be, for example, a laser, such as, a mode locked Ti:Al₂O₃ laser, one or more diodes, including but not limited to, a light emitting diode (LED) such as an edge emitting diode, multiple quantum well emitting diodes and a superluminescent diode (SLD), a white light source, electromagnetic (EM) wave sources in different frequency and wavelength ranges, superfluorescent optical fibers, and the like. The light source may further include one or more light sources having the same or different wavelengths, or may include one or more quantum well devices formed on a single substrate to provide light at multiple wavelengths. Light emitted by a light source such as those described above may be emitted at near infrared or infrared wavelength, have short coherence length and may have high irradiance for penetrating deep into the sample and may include, but not be limited to, low coherence light or multiple low coherence light having different center wavelengths whose outputs have been combined. In general, low coherence light may have wavelengths of about at least 600 nm. The penetration of the light into the sample may vary depending on, for example, the wavelength and power of the source light used, the presence of optical circulators, coupling losses, component attenuation light, the sample type and so on, and may be capable of penetrating a sample and providing backscattered ballistic light as well as non-ballistic light traveling in torturous trajectories through the sample.

The waveguides utilized in embodiments of the invention may be of any type known in the art such as, for example, optical emitting fibers, including, single mode (SM) or polarization-maintaining (PM) optical fibers. As used herein, “optical emitting fibers” refers to optical fibers that are typically made of glass or a material having a higher dielectric constant than the surrounding medium. An optical emitting fiber generally has a core and a cladding. By core is meant the part of the optical fiber through which light is guided, and the choice of core size depends on the wavelength and numerical aperture, and on whether the fiber is intended to propagate light as a single waveguide mode or several waveguide modes. Typically, single-mode fiber core sizes for wavelengths in the visible and near infra-red range may be about 5 to about 9 microns in diameter. Cladding is of a material having a lower refractive index than the core material and may surround the core to both ensure light guiding as well as to add mechanical strength to the fiber. The core and cladding of an optical fiber may be composed of any material through which light may pass including, but not be limited to glass, polymers, plastics, and combinations thereof.

The optical probes of embodiments may further include any type of optical shaping or redirecting device known and useful in the art optically coupled to either the light source or one or more waveguide. Such devices include, but are not limited to, light splitters, optical couplers or light combiners, fiber couplers, optical circulators, prisms, mirrors, lenses, holographic elements, polarizers, polarization controllers, optical delays, drive motors, movable mirrors, optical stretchers and variable optical attenuators. For example, in a typical Michelson interferometer, light from a light source may be propagated to a light splitter where light is directed to at least one reference arm and one or more sample arm. The splitter may be operative to both split the optical power of the light source for propagation through the reference and sample arms of the interferometer and combine backscattered light from the sample with light from the reference arm. The optical power may be split equally or unequally.

In various embodiments, the optical probes may run the length of the ablation catheter from a proximal position which is maintained outside of the lumen or a patient under examination to the distal most catheter tip, and the arrangement and attachment site of the optical probe may vary. For example, in some embodiments, a single optical probe may be attached to the outermost shell of the ablation catheter, and in others, a single optical probe may be embedded within the catheter nearer the effectors. In still other embodiments, multiple optical probes may be arranged around the outermost shell of the catheter or embedded within the ablation catheter, and in certain embodiments, one or more optical probes may be attached to the outermost shell and one or more optical probe may be embedded within the catheter.

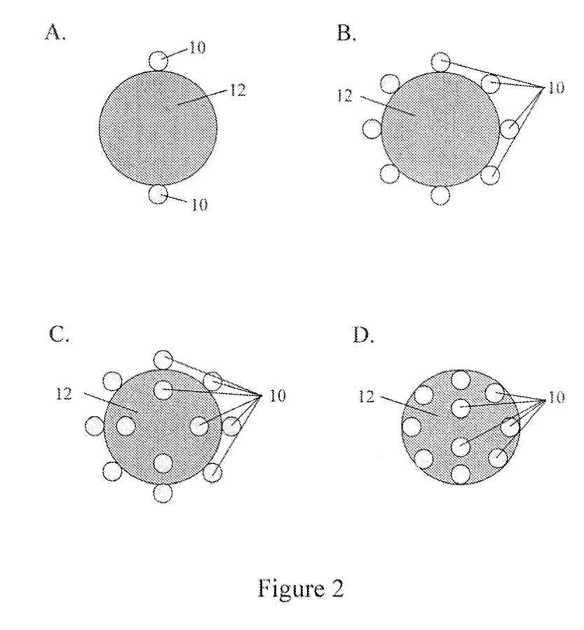

The optical probes may be arranged in any way. For example, in some embodiments, as illustrated in FIG. 2, Panel A, one or more optical probes 10 may be arranged on an upper portion of an ablation catheter 12, and/or one or more optical probes 10 may be arranged on a lower portion of the catheter 12. In other embodiments, as illustrated in FIG. 2, Panel B, a plurality of optical probes 10 may be equally spaced around an external circumference of the catheter 12. In still other embodiments, as illustrated in FIG. 2, Panel C, a plurality of optical probes 10 may be equally spaced around an external circumference of the catheter 12 and a plurality of optical probes 10 may be embedded within the catheter 12. In yet other embodiments, as illustrated in FIG. 2, Panel D, a plurality of optical probes 10 may be embedded within the catheter 12.

The optical probes may emit light at any angle. For example, in some embodiments, the optical probes associated with a catheter may emit light that is parallel to the central axis of the catheter. Therefore, light form an optical probe is emitted directly into tissue contacted by energy from the ablation catheter. In other embodiments, light is emitted from the optical probe at one or more angle such as, for example, an angel perpendicular to the central axis of the catheter, and in certain embodiments, a combination of optical probes emitting light parallel to the central axis of the catheter and at an angle other than parallel to the central axis of the catheter may be used. Without wishing to be bound by theory, the ability to collect an optical signal from various angles surrounding the catheter may aid in aligning the catheter at an appropriate position on the target.

Backscattered light from the sample may be propagated to a receiver in various embodiments of the invention. Briefly, a receiver detects the backscattered light, converts the light signal to an electrical, analog and/or digital signal and transmits the signal to a processor. Receiver architectures may vary among embodiments and may depend on the type of signal received by the receiver or the input of the processor. A receiver may include any number of components, such as, but not limited to, optical couplers, optical splitters, optical circulators, amplifiers, polarization controllers, detectors, digital acquisition boards, and processors coupled to one another in a multitude of arrangements. Once the data has been processed the results may be displayed on any output device known in the art, such as, for example, a monitor or printout.

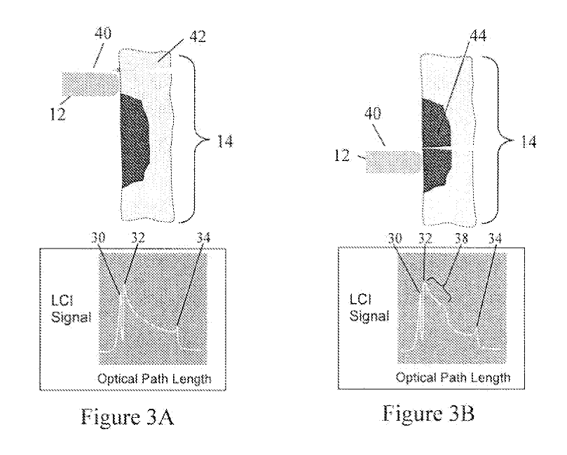

FIG. 3 illustrates a typical LCI output associated with the detection of an area of ablation on a target tissue from a Michelson type interferometer consisting of a reference arm (not shown) and single sample arm probe 40 mounted on the top of an ablation catheter 12. Signal may be collected from various depths within the interrogated tissue 14 by adjusting the optical path length of the reference light in a process known as “scanning”. During scanning, the length of the reference arm is increased using for example, an optical delay line, optical stretcher, or a movable mirror and the probed depth of the sample is increased to a depth corresponding with the length of the reference arm. Therefore, the measurement of the peak gating function gives the amplitude of the profile of the signal as a function of depth (inset FIG. 3A and FIG. 3B), and the data collected during scanning may be used to identify irregular structures up to a specific depth corresponding with the maximum length of the reference arm in the sample. For example, the inset of FIG. 3A shows the depth profile of normal tissue 42. The fiber tip 30, edge of front wall of the tissue under interrogation 32 and rear wall of the tissue under interrogation 34 can be readily discerned from each scan. The presence of ablated tissue 44 may also be observed from the depth profile of the tissue scan as illustrated in the inset of FIG. 3B. As with normal tissue, the fiber tip 30, edge of front wall of the tissue under interrogation 32 and rear wall of the tissue under interrogation 34 can be discerned from a scan of ablated tissue along with an extension of the peak caused by the front wall of the tissue 38 or secondary peaks arising at longer pathlengths are indicative of ablated tissue. Methods and techniques for acquiring interferometric data and using such data to interrogate tissue are well known in the art. For example, see U.S. Pat. No. 7,184,148 entitled “Low Coherence Interferometry Utilizing Phase” and U.S. Pat. No. 7,190,464 entitled “Low Coherence Interferometry for Detecting and Characterizing Plaques” hereby incorporated by reference in their entireties. The length of the reference arm may be adjusted over any number of increments during scanning. For example, a “quick” scan may be performed by adjusting the length of the reference arm by large increments, and a continuous scan carried out using very small increments may be used to precisely define structures identified using a quick scan.

Various embodiments of the invention may further include a balloon or other such device on the exterior of the ablation catheter. Such a balloon may be part of a separate device provided over the catheter or may be built into the interior of the ablation catheter. Balloons may be prepared from any material known in the art including hard or semi-hard glass, plastic, rubber, or other transparent material and must be capable of withstanding the penetration of both light from the optical probes and the energy from the ablation catheter. Such balloons may be inflated using any liquid or gas through which an optical signal may be passed without scattering and may be inflated to a fixed volume provided that its diameter and flexibility are sufficient for navigation through blood vessels and the heart to the location of the target tissue. Without wishing to be bound by theory, the balloon may also provide a soft envelope which may prevent unintended injury and/or physical damage to either the catheter or the patient during manipulation of the catheter during use.

The invention described herein also encompasses methods for using an ablation catheter having one or more optical probes such as those of embodiments described above. In various embodiments, the ablation catheter having one or more optical probe may be provided to a target area, such as, for example, an atria of a heart; the catheter may be aligned with a site of ablation within the target area, such as, an ectopic focus; the ablation catheter may be activated; and energy may be applied to the site of ablation initiating treatment. The optical probes associated with the ablation catheter may be activated concurrently with the ablation catheter and continuously monitor the target area such that when injury induced by the application of the energy has reached a specific depth, application of the energy is terminated. In some embodiments, the method may further include confirming that the injury induced by the application of energy has reached the appropriate depth following termination of the energy, by continuing to monitor the target area or site of ablation following ablation. Embodiments of the method also include examining the target area and site of ablation prior to the application of energy to identify irregularities in the target area, such as, for example, previous injury or previous sites of ablation, to avoid unintended injury to a patient that might occur if treatment is applied to these areas. The method may also include using the optical probes to ensure that the ablation catheter is properly aligned. For example, the distance between the catheter and the site of ablation may be determined using the optical probe to ensure that each optical probe in an ablation catheter having multiple optical probes is the same distance from the site of ablation and, therefore, the catheter is substantially perpendicular to the site of ablation.

In various embodiments, a site of ablation may be previously determined using conventional techniques. However, the optical probes associated with the catheter may be used to ensure proper placement of the catheter and/or to identify site of ablation. For example, probes which emit light perpendicular to the axis of the catheter may be used to interrogate the target area and/or identify structures within the target area. When the site of ablation is identified, the en-face probes may be concurrently utilized with perpendicular probes to determine the angle at which the catheter is facing the site of ablation, because both en-face and perpendicular probes may provide a signal. As the catheter takes on a more perpendicular position with respect to the site of ablation, signal from the perpendicular probe may be lost as signal from the en-face probe continues to produce signal. In this way, the position of the catheter with respect to the site of ablation may be continuously monitored during deployment and retraction of the catheter and during treatment.

In various embodiments, the predetermined depth may be depths calculated to ablate the tissue encompassing and/or surrounding a site of ablation but not a depth such that the tissue of the target area, such as, an atrium of the heart, is breached. In some embodiments, the predetermined depth may be decided by a user who observes the data collected by the optical probes and terminates treatment when a suitable depth of ablation has been reached and before non-target or collateral tissues or structures have been damaged. In other embodiments, the predetermined depth may be calculated based on general knowledge and/or previous tests performed on the patient, and this value may be entered into a processor. The processor may then control the administration of treatment and automatically terminate treatment when the predetermined depth is reached.

In other embodiments, information regarding the extent of injury collected by the optical probes may be used as part of a feed-back loop wherein, for example, treatment is terminated when the predetermined maximum depth is reached or when an irregularity, such as loss of signal from one or more probe occurs. Such feed-back loops may be controlled by a processor programmed to receive data from the optical probe and determine the depth of ablation. Thus, unintended injury due to over exposure to treatment may ba avoided. In yet another embodiment, user may monitor the depth of ablation at the same time as a processor.

In still other embodiments of the method, the site of ablation may be reevaluated using the optical probes following administration of treatment. For example, treatment may be terminated when ablation has reached an appropriate depth, and the optical probes may be used to ensure that ablation has occurred to the predetermined depth over the entire site of treatment. Further treatment may then be applied if additional ablation is required or desired. In another example, the optical probes along with other sensors associated with the ablation catheter may be used to survey and evaluate the site of ablation to determine whether, for example, abnormal electrical impulses associated with an ectopic focus persist. In the event that the abnormal impulses persist, the optical proves may be used to ensure that a secondary site of ablation is free of injury and a safe distance from the previous site of ablation or from non-targeted or collateral tissues and structures.

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. An apparatus comprising:
an ablation catheter; and
at least one optical probe wherein said at least one optical probe monitors a depth of ablation during use of the ablation catheter.

2. The apparatus of claim 1, wherein low coherence interferometry is used to monitor the depth of ablation.

3. The apparatus of claim 1, wherein the at least one optical probe is mounted to an exterior circumference of the ablation catheter, embedded in the ablation catheter, or combinations thereof.

4. The apparatus of claim 1, wherein the at least one optical probe delivers and collects light in a plane parallel to a central axis of the ablation catheter.

5. The apparatus of claim 1, wherein the at least one optical probe delivers and collects light in a plane perpendicular to a central axis of the ablation catheter.

6. The apparatus of claim 1, wherein the at least one optical probe delivers and collects light in more than one plane.

7. The apparatus of claim 1, wherein the at least one optical probe is contained in a housing other than the ablation catheter.

8. The apparatus of claim 7, wherein the at least one optical probe is a guidewire probe.

9. The apparatus of claim 1, wherein the ablation catheter delivers ablation energy selected from electrical energy, RF energy, ultrasound energy, optical, infrared, microwave, laser light, cryogenic energy and combinations thereof.

10. The apparatus of claim 1, further comprising a balloon disposed on an exterior surface of the ablation catheter.

11. A method for monitoring ablation comprising:
providing an ablation catheter and at least one optical probe to a target area wherein said at least one optical probe monitors a depth of ablation during use of the ablation catheter;
identifying a site of ablation within the target area;
applying energy to the site of ablation to cause ablation; and
monitoring a depth of ablation concurrently with the application of energy.

12. The method of claim 11, wherein the energy is selected from electrical energy, RF energy, ultrasound energy, optical, infrared, microwave, laser light, cryogenic energy and combinations thereof.

13. The method of claim 11, wherein the step of monitoring uses low coherence interferometry.

14. The method of claim 11, wherein step of monitoring the depth of ablation occurs in real-time.

15. The method of claim 11, wherein the step of monitoring uses at least one optical probe that is contained in a housing other than the ablation catheter.

16. The method of claim 15, wherein the at least one optical probe is a guidewire probe.

17. The method of claim 11, further comprising terminating the application of energy when the injury has reached a predetermined depth.

18. The method of claim 17, wherein the application of energy is terminated by a user.

19. The method of claim 17, wherein the application of energy is automatically terminated by a processor based on a feed-back loop controlled by the processor.

20. The method of claim 17, further comprising:
interrogating the site of ablation following the termination of the application of energy; and
confirming that ablation has occurred.

21. The method of claim 11, further comprising:
interrogating the site of ablation prior to applying energy; and
identifying irregularities at the site of ablation.

22. The method of claim 11, wherein the ablation occurs in cardiac tissue.

23. The method of claim 11, wherein the ablation is used to treat an ectopic focus.

24. A system for monitoring ablation comprising:
an ablation catheter;
at least one optical probe;
a receiver configured to receive a signal from the at least one optical probe
a processor configured to determine the depth of the ablation in real-time during ablation; and
an output device for displaying the depth.

25. The system of claim 24, wherein the at least one optical probe is used to perform low coherence interferometry.
